(19) United States
(12) Patent Application Publication
(10) Pub. No.: US 2011/0135755 A1
(43) Pub. Date: Jun. 9, 2011

(54) Combination therapies for treating neoplastic disease

(76) Inventors: Roger Anthony Oakes, Greetham (GB); Andor Sebasteny, Grimsby (GB); Loannis Papasotiriou, Filotas (GR); Janet Nicola Hembry, Bristol (GB)

(21) Appl. No.: 12/654,047

(22) Filed: Dec. 8, 2009

(51) Int. Cl. A61K 33/24; A61P 35/00; A61K 31/60; A61K 33/04

(52) U.S. Cl. 424/649; 514/161; 424/705

(57) ABSTRACT

A method for treating neoplastic disease in a patient is described, which comprises administering to the patient: an antineoplastic platinum (II) complex; a physiologically acceptable source of assimilable copper; a physiologically acceptable source of assimilable manganese; a source of salicylic acid or a physiologically acceptable derivative thereof; and vitamin C.

### BACKGROUND OF THE INVENTION

This invention relates to combination therapies for treating neoplastic disease.

Various classes of antineoplastic agent are known, major types including: nitrogen mustards; ethyleneimine compounds; alkyl sulfonates; platinum(II) complexes; vinca alkaloids; taxanes; podophyllotoxin derivatives; camptothecin derivatives; and certain antibiotics. In certain instances a combination chemotherapy regimen may be chosen, wherein more than one type of chemotherapeutic agent is administered, the choice of antineoplastic agents depending on the type of neoplasm being treated, spectrum of activity and interactions of the drug, and other clinical considerations. However, due to the highly toxic nature of most chemotherapeutic agents and the potential for adverse interactions to occur, care is needed when adopting a combination chemotherapy regimen and in choosing the antineoplastic agents to be combined.

As noted above, one type of known antineoplastic agent are the platinum (II) complexes. Examples include cisplatin, carboplatin, oxaliplatin, nedaplatin, satraplatin, lobaplatin and heptaplatin.

Cisplatin (cis-diamminedichloroplatinum) is the most often used platinum (II) complex. It is used in the management of many solid malignancies, including in particular those of the bladder, cervix, lung, ovary, and testis. Other malignancies where cisplatin may be employed include non-Hodgkin's lymphomas, tumours of the brain, endometrium, oesophagus, stomach, anus, head and neck, and thymus, neuroblastomas, and sarcoma of the bone and soft tissue.

However, cisplatin does have a significant toxicological profile. Severe nausea and vomiting occur in most patients during treatment. Serious toxic effects on the kidneys (nephrotoxicity), bone marrow and ears (ototoxicity) have been reported in up to one third of patients given a single dose, with effects generally being both dose-related and cumulative. Nephrotoxicity is, in particular, a well-established adverse effect of cisplatin, and can be dose limiting. In addition to the above, neurological effects (additional to ototoxicity) such as peripheral neuropathies, loss of taste, seizures, and ocular toxicities have also occurred, as have anaphylactoid reactions and cardiac abnormalities. Moreover, cisplatin, like other platinum derivatives, is potentially mutagenic and teratogenic.

Infusion of cisplatin with an osmotic diuretic such as mannitol, and hydration with chloride-containing solutions before and after treatment, has been shown to reduce nephrotoxicity, and this is therefore a now a standard practice. Various other measures for reducing the toxicity of cisplatin have also been suggested or investigated in the scientific literature. Addition of magnesium to pre- and post-hydration fluids to combat renal-magnesium wasting has been suggested. Sulfhydryl metabolism and oxidative stress appear to play a role in toxicity, and measures that reduce glutathione depletion and scavenge intracellular free oxygen radicals have been tried in an attempt to modulate nephrotoxicity. Sulfur-containing nucleophiles, amifostine, glutathione and sodium thiosulphate have been investigated for their chemopreventative potential. Various substances, including thiols, amifostine, ebselen, allopurinol, salicylates, vitamin E, and glutathione, have be investigated for protective effects against neurotoxicity.

WO-A-01/24802 teaches compositions for use in the treatment or prophylaxis of neoplastic disease. The compositions comprise at least: a physiologically acceptable source of assimilable copper and manganese; a source of salicylic acid or a physiologically acceptable derivative thereof; and vitamin C. Optional additional components comprise: a physiologically acceptable source of assimilable manganese; a physiologically acceptable source of assimilable iron; a physiologically acceptable source of assimilable sulfur; and a physiologically acceptable source of assimilable zinc. Examples are provided demonstrating the use of compositions, comprising copper orotate/gluconate, sodium salicylate, manganese orotate/gluconate, and vitamin C, in the treatment of various tumours in various animal studies.

A composition under the name of CV247 has undergone further investigations as an anti-neoplastic agent, including vetinary studies and phase (II) clinical trials. CV247 is a composition is in accordance with the teaching of WO-A-01/24802, and consists of an orally administered aqueous solution of manganese gluconate (2 mg/ml), copper gluconate (2 mg/ml), vitamin C (40 mg/ml) and sodium salicylate (35 mg/ml).

### BRIEF SUMMARY OF THE INVENTION

It has now unexpectedly been discovered that compositions comprising an assimilable copper compound, an assimilable manganese compound, a source of salicylic acid or a derivative thereof, and vitamin C, have surprising and synergistic effects when used in combination with an antineoplastic platinum (II) complex. More particularly, it has now been discovered that administering such a composition alongside an antineoplastic platinum (II) complex both significantly increases the level and period of efficacy of antineoplastic therapy and significantly reduces the level of nephrotoxicity. The enhanced efficacy of the combined treatment, as compared to administration of the antineoplastic platinum (II) complex on its own, in addition allows for administration of a significantly reduced dosage of antineoplastic platinum (II) complex, leading to further consequential reductions in dose related toxic side effects.

Accordingly, in a first aspect the present invention provides a method for treating neoplastic disease in a patient, comprising administering to the patient: (a) an antineoplastic platinum (II) complex; (b) a physiologically acceptable source of assimilable copper; (c) a physiologically acceptable source of assimilable manganese; (d) a source of salicylic acid or a physiologically acceptable derivative thereof; and (e) vitamin C.

In a second aspect, the invention provides a method for enhancing the antineoplastic effect (enhancing, for example, the level of antineoplastic effect and/or the period of efficacy) of an antineoplastic platinum (II) complex and/or for reducing the toxic side effects (such as nephrotoxicity and/or other known toxic effects) of an antineoplastic platinum (II) complex in a patient receiving said antineoplastic platinum (II) complex. The method comprises administering to the patient (before, after, and/or during administration of the antineoplastic platinum (II) complex): a physiologically acceptable source of assimilable copper; a physiologically acceptable source of assimilable manganese; a source of salicylic acid or a physiologically acceptable derivative thereof; and vitamin C.

In a third aspect, the invention provides an antineoplastic therapeutic combination (which may be a single pharmaceutical composition, or may be a combination of two or more compositions for simultaneous or sequential administration, for example in the form of a kit) comprising: (a) an antineoplastic platinum (II) complex; (b) a physiologically acceptable source of assimilable copper; (c) a physiologically acceptable source of assimilable manganese; (d) a source of salicylic acid or a physiologically acceptable derivative thereof; and (e) vitamin C.

### BRIEF DESCRIPTION OF THE DRAWINGS

FIG. 1 is a bar chart depicting the free SH-group concentration in the plasma of rats treated with cisplatin, CV247 (a composition comprising copper gluconate, manganese gluconate, sodium salicylate, and vitamin C), or both cisplatin and CV247;

FIG. 2 is a bar chart depicting the free SH-group concentration in the liver homogenate from rats treated with cisplatin, CV247, or both cisplatin and CV247;

FIG. 3 is a bar chart depicting free-radical induced chemiluminescence in the plasma of rats treated with cisplatin, CV247, or both cisplatin and CV247;

FIG. 4 is a bar chart depicting free-radical induced chemiluminescence in the liver homogenate from rats treated with cisplatin, CV247, or both cisplatin and CV247;

FIG. 5 is a bar chart depicting the level of diene conjugates in the liver homogenate from rats treated with cisplatin, CV247, or both cisplatin and CV247;

FIGS. 6A and B are graphs depicting the results of an MTT assay, studying the effects of varying concentrations of cisplatin and CV247 on an HCT 8 cell line using, respectively, medium (18000 cells/well) and high (26000 cells/well) cancer cell burdens;

FIGS. 7A and B are graphs depicting the results of an SRB assay, studying the effects of varying concentrations of cisplatin and CV247 on an HCT 8 cell line using, respectively, medium (18000 cells/well) and high (26000 cells/well) cancer cell burdens;

FIGS. 8A and B are graphs depicting the results of an CVE assay, studying the effects of varying concentrations of cisplatin and CV247 on an HCT 8 cell line using, respectively, medium (18000 cells/well) and high (26000 cells/well) cancer cell burdens;

FIGS. 9A and B are graphs depicting the results of an MTT assay, studying the effects of varying concentrations of cisplatin and CV247 on an T47D cell line using, respectively, medium (18000 cells/well) and high (26000 cells/well) cancer cell burdens;

FIGS. 10A and B are graphs depicting the results of an SRB assay, studying the effects of varying concentrations of cisplatin and CV247 on an T47D cell line using, respectively, medium (18000 cells/well) and high (26000 cells/well) cancer cell burdens; and

FIGS. 11A and B are graphs depicting the results of an CVE assay, studying the effects of varying concentrations of cisplatin and CV247 on an T47D cell line using, respectively, medium (18000 cells/well) and high (26000 cells/well) cancer cell burdens.

### DETAILED DESCRIPTION OF THE INVENTION

As noted above, the present invention in one aspect concerns a method for treating neoplastic disease in a patient comprising administering to the patient: (a) an antineoplastic platinum (II) complex; (b) a physiologically acceptable source of assimilable copper; (c) a physiologically acceptable source of assimilable manganese; (d) a source of salicylic acid or a physiologically acceptable derivative thereof; and (e) vitamin C. In another aspect, the present invention concerns an antineoplastic therapeutic combination comprising compounds (a)-(e). In yet another aspect, the invention concerns a method that comprises administering compounds (b)-(e) to a patient receiving compound (a) so as to enhance the antineoplastic effect and/or reduce the toxic side effects of compound (a).

In some embodiments, one or more of the following compounds are also administered to the patient or are also present in the therapeutic combination: (f) a physiologically acceptable source of assimilable iron; (g) a physiologically acceptable source of assimilable zinc; and (h) a physiologically acceptable source of assimilable sulfur.

The antineoplastic platinum (II) complex may be of any type known in the art. Preferred antineoplastic platinum (II) complexes include cisplatin, oxaliplatin and carboplatin. Cisplatin is particularly preferred. More than one type of antineoplastic platinum (II) complex may also be employed in the methods and/or therapeutic combinations of the present invention. For example, combinations of cisplatin and carboplatin may be used.

The sources of copper, manganese, iron and zinc used in the present invention preferably contain the metals in ionic form, e.g. as salts with organic or inorganic acids. However, other metal compounds which provide assimilable sources of the metals, e.g. metal oxides, can also be used.

Thus, a physiologically acceptable source of assimilable copper is typically a copper oxide or a salt of copper with an organic or inorganic acid. A physiologically acceptable source of assimilable manganese is typically a manganese oxide or a salt of manganese with an organic or inorganic acid. A physiologically acceptable source of assimilable iron is typically an iron oxide or a salt of iron with an organic or inorganic acid. A physiologically acceptable source of assimilable zinc is typically a zinc oxide or a salt of zinc with an organic or inorganic acid.

Suitable physiologically acceptable salts of the above metals with organic acids include salts with orotic acid, aspartic acid, gluconic acid, tartaric acid, citric acid, lactic acid, acetic acid, fumaric acid, maleic acid, malic acid, ascorbic acid, succinic acid, benzoic acid, methanesulphonic acid, ethanesulphonic acid, benzenesulphonic acid and p-toluenesulphonic acid. Suitable physiologically acceptable salts of the above metals with inorganic acids include salts with hydrochloric acid, hydrobromic acid, hydriodic acid, phosphoric acid, diphosphoric acid, nitric acid or sulfuric acid, preferably hydrochloric, hydrobromic, hydroiodic, phosphoric or sulfuric acid. Such salts are available commercially or may be prepared if desired by known methods.

Preferred physiologically acceptable salts are salts with organic acids, more preferably salts with orotic acid, aspartic acid, gluconic acid, tartaric acid, citric acid, lactic acid or acetic acid and most preferred are salts with orotic or gluconic acid.

It is also preferred that the physiologically acceptable salts are water soluble, for example salts with gluconic acid.

It is particularly preferred that the physiologically acceptable salt of assimilable copper is copper orotate or copper gluconate, most preferably copper gluconate. It is particularly preferred that the physiologically acceptable salt of assimilable manganese is manganese orotate or manganese gluconate, most preferably manganese gluconate. It is particularly preferred that the physiologically acceptable salt of assimilable iron is iron orotate or iron gluconate, most preferably iron gluconate. It is particularly preferred that the physiologically acceptable salt of assimilable zinc is zinc orotate or zinc gluconate, most preferably zinc gluconate.

When, as is preferred, two or more of the source of assimilable copper, source of assimilable manganese, source of assimilable iron, and source of assimilable zinc are used on the form of a metal salt, all the metal salts preferably include the same anion. This anion is typically orotate or gluconate, preferably gluconate.

The source of salicylic acid or a physiologically acceptable derivative thereof is typically salicylic acid or a physiologically acceptable derivative thereof. Typically, the said derivative is a compound in which the carboxyl or hydroxyl function of salicylic acid has been converted into a derivative.

A physiologically acceptable derivative of salicylic acid is typically a salicylic acid metal salt, ester or amide. Examples of suitable metal salts include alkali metal salts, for example sodium and potassium salts, and alkaline earth metal salts, for example calcium and magnesium salts. Sodium salicylate is most preferable.

Examples of suitable esters include C₁₋₆ alkyl esters, for example methyl, ethyl, propyl, butyl, pentyl or hexyl esters and particularly preferred are the methyl and ethyl esters. Examples of suitable amides are amides obtainable by reacting salicylic acid with an amine HNR₁R₂, wherein R₁ and R₂ may be the same or different and are selected from hydrogen and C₁₋₆ alkyl groups such as methyl, ethyl, propyl, butyl, pentyl or hexyl. R₁ and R₂ are preferably selected from hydrogen, methyl and ethyl and most preferably both R₁ and R₂ are hydrogen.

Derivatives in which both the hydroxyl function and the carboxyl function of salicylic acid have been converted into a derivative can also be used. When the hydroxyl function of salicylic acid is converted to a derivative it is typically converted to an ester, for example a C₁-C₆ alkyl ester such as acetyl-salicylic acid (aspirin).

A particularly preferred derivative of salicylic acid is sodium salicylate. Salicylic acid itself and suitable derivatives of it are commercially available.

Typically, the physiologically acceptable source of assimilable sulfur is elemental sulfur and any allotropic form of sulfur may be used. Preferably, sulfur is present in the composition in the form of sublimed sulfur or precipitated sulfur, most preferably sublimed sulfur.

In the methods and therapeutic combinations of the present invention, compounds (a)-(e) and, where present, (f), (g) and/or (h), may be formulated as a single composition (for example they could all be present in a single composition for intravenous infusion), or they may be formulated as two or more separate compositions (for example where each compound is present in a separate composition, or where two or more but not all of the compounds are present in one composition and the remainder are present in one or more separate compositions).

In one preferred embodiment, compound (a) is formulated and administered separately from compounds (b)-(e) and, optionally and if used, (f), (g) and/or (h).

It is preferred that compounds (b)-(e) and, optionally and if used, (f), (g) and/or (h), are formulated and administered as a single composition. Compounds (b)-(e) and, if present, (f)-(h) may be the sole pharmaceutically active components of the composition, or other pharmaceutically active components may be present.

Where one or more of the compounds are formulated as a separate composition or compositions from the remainder of the compounds, the compounds may, as noted above, be administered separately. The compounds may be administered via the same or different routes of administration, and may be administered simultaneously or sequentially. For example, in one embodiment of the invention, compound (a) is administered via intravenous infusion, and compounds (b)-(e) and, if used, (f), (g) and/or (h) are administered as an oral composition. It is preferred that compounds (a)-(e) and, where used, (f)-(h), are administered concomitantly or sequentially, although the duration of administration of the compounds may vary, such that, for example, the administration of (a) may commence before and/or end after administration of (b)-(e) and, where used, (f)-(h).

Where compounds (b)-(e) are formulated as a single composition, the composition typically comprises these compounds in the following amounts:

15 to 60, preferably 25 to 40, parts by weight copper gluconate, or equivalent amount of active ingredient when a physiologically acceptable source of assimilable copper other than copper gluconate is used;

15 to 60, preferably 25 to 40, parts by weight manganese gluconate, or equivalent amount of active ingredient when a physiologically acceptable source of assimilable manganese other than manganese gluconate is used

300 to 600, preferably 300 to 400, most preferably 350, parts by weight sodium salicylate, or equivalent amount of active ingredient when salicylic acid or a physiologically acceptable derivative thereof other than sodium salicylate is used; and

200 to 1000, preferably 300 to 500, most preferably 400, parts by weight vitamin C, vitamin C preferably being present in the compositions of the invention in an amount significantly larger than that which is regarded as the normal minimum daily requirement for an adult.

Where one or more of compounds (f)-(h) are to be used, and are to be formulated in the same composition as that comprising compounds (b)-(e), the composition typically comprises these further compounds in the following amounts:

15 to 60, preferably 25 to 40, parts by weight iron gluconate, or equivalent amount of active ingredient when a physiologically acceptable source of assimilable iron other than iron gluconate is used;

15 to 60, preferably 25 to 40, parts by weight zinc gluconate, or equivalent amount of active ingredient when a physiologically acceptable source of assimilable zinc other than zinc gluconate is used; and

15 to 60, preferably 25 to 40, parts by weight sulfur.

The parts by weight referred to above are based on the total weight of these ingredients in the composition.

In the present invention, the amount, frequency and route of administration of compound (a) may be in accordance with the amounts, frequency and routes via which said compound is conventionally administered. For example, cisplatin is often administered, via intravenous infusion, in a single dose of about 50 to 120 mg/m² every 3 to 4 weeks. Alternatively, about 15 to 20 mg/m² may be given daily for 5 days, every 3 to 4 weeks. The intravenous infusion solution employed may, for example, be an aqueous sodium chloride (e.g. 0.9%) or sodium chloride and glucose solution, and may comprise other compounds, such as mannitol (e.g. 375 ml of mannitol 10%).

In other embodiments the amount of compound (a) administered may be less than that conventionally administered, due to the enhancement in antineoplastic effect provided by compounds (b)-(e). For example, in certain embodiments, the dosage of compound (a) administered may be as little as about a fifth (⅕th) of its normally administered dosage. Thus, where compound (a) is cisplatin it may, for example, be administered (preferably via intravenous, intra-arterial or intraperitoneal infusion, most preferably via intravenous infusion) in a single dose of about 10 to 25 mg/m² every 3 to 4 weeks, or in doses of about 3 to 4 mg/m² given daily for 5 days, every 3 to 4 weeks.

Suitable formulations of cisplatin and other antineoplastic platinum (II) complexes are commercially available, and can be readily obtained.

The dosage of compounds (b)-(e) and, if used (f)-(h), administered in the methods of the present invention should be calculated having regard to the weight of the patient. Where these compounds are to be administered orally in a single composition, an exemplary dosage of the composition would be about 2 ml volume for each 60 lbs of body weight of the subject to be treated, which dosage can be administered up to three times a day. The 2 ml volume dosage typically contains from 8 to 35 mg, preferably from 14 to 25 mg of copper gluconate, or an equivalent amount of active ingredient when a physiologically acceptable source of copper other than copper gluconate is used. The 2 ml volume dosage typically contains from 8 to 35 mg, preferably from 14 to 25 mg of manganese gluconate or an equivalent amount of active ingredient when a physiologically acceptable source of manganese other than manganese gluconate is used. The 2 ml volume dosage typically contains from 170 to 350 mg, preferably from 170 to 230 mg and most preferably about 200 mg sodium salicylate or an equivalent amount of active ingredient when salicylic acid or a physiologically acceptable derivative thereof other than sodium salicylate is used. The 2 ml volume dosage typically contains from 110 to 570 mg, preferably from 170 to 285 mg and most preferably about 230 mg vitamin C.

A suitable dosage of about 2 ml volume of a composition further comprising a physiologically acceptable source of assimilable iron typically contains from 8 to 35 mg, preferably from 14 to 25 mg of iron gluconate or an equivalent amount of active ingredient when a physiologically acceptable source of iron other than iron gluconate is used.

A suitable dosage of about 2 ml volume of a composition further comprising a physiologically acceptable source of assimilable zinc typically contains from 8 to 35 mg, preferably from 14 to 25 mg of zinc gluconate or an equivalent amount of active ingredient when a source of zinc other than zinc gluconate is used.

A suitable dosage of about 2 ml volume of a composition further comprising a physiologically acceptable source of assimilable sulfur typically contains from 8 to 35 mg, preferably from 14 to 25 mg of sulfur.

These figures are approximate and considerable variation in the proportions of the active ingredients is possible without losing the valuable properties of the compositions.

The compositions comprising compounds (b)-(e) and, optionally, (f)-(h) may be made by first forming an intimate mixture of the metals to be used in the form of suitable salts or other derivatives, together with sulfur, if present. This mixture in finely ground form can then be added to an aqueous solution or suspension of the salicylic acid or derivative thereof. Typically, from 2 to 5 ml, preferably about 3½ ml of aqueous solution or suspension is used. This solution preferably contains 5-20%, preferably about 10%, by weight of salicylic acid or derivative. The vitamin C may be added before or after the salicylic acid solution, and is preferably added before the salicylic acid solution such that all of the solid ingredients are combined first. The resulting slurry or solution may be administered orally.

As noted above, an exemplary dosage of the composition, comprising active ingredients in the amounts set out above, in the form of an aqueous solution or suspension, is 2 ml per 60 lbs body weight of subject. An initial dosage of this amount may, for example, be followed by a half dose of a similar solution or suspension 1 to 2 hours later. Four hours later a further half dose may be given. Subsequent treatment (when the tumour has noticeably regressed and/or the symptoms have been considerably alleviated) may consist of the oral administration of 2 ml of the said solution or suspension per 60 lbs body weight of subject once a day. This may be given for three weeks, then, if further progress has been made, the dose may be reduced to 2 ml per 60 lbs body weight on alternate days for 3 weeks. The frequency of dosing may be further reduced as further progress is made.

The methods and therapeutic combinations of the present invention may be used in human and veterinary medicine, for example in the treatment of humans, cats or dogs.

As noted above, it has been found that when compounds (b)-(e), and optionally (f)-(h), are administered, preferably concomitantly, with an anti-neoplastic platinum (II) complex, such as cisplatin, there is a significant increase in both the level of effect and the period of effectiveness of said platinum containing drugs, with regard to cancer cell “kill” or reduced viability, as compared to the level of effect and period of efficacy when said platinum containing drug is administered on its own. As the same level of effect can, therefore, be attained with a considerably reduced dose of the platinum containing drug, and as compounds (b)-(e) also have a direct effect in reducing at least the nephrotoxicity of the platinum containing drug, dose related side effects of the platinum containing drug, including nephrotoxicity, can in addition be minimised.

The methods and therapeutic combinations of the invention may be used for the treatment of a variety of neoplastic diseases, including any and all of those for which the antineoplastic platinum (II) complex is indicated. In certain embodiments of the invention, the neoplastic disease to be treated is selected from solid malignancies (including, in particular, those of the bladder, cervix, lung, ovary, and testis), tumours of the brain, endometrium, oesophagus, stomach, anus, head and neck, and thymus, neuroblastomas, sarcoma of the bone and soft tissue, carcinomas of the breast, rectum, colon, prostate, bladder, liver, peritoneum, stomach and urethra, and certain other lymphomas and sarcomas.

The compositions comprising compounds (b)-(e) and, optionally, (f)-(h), are, as noted above, normally administered orally. Preferably, therefore, they are formulated so as to be suitable for oral administration. Suitable forms for oral administration include, for example, tablets, troches, lozenges, aqueous or oily suspensions, dispersible powders or granules. Preferred forms for oral administration are tablets and capsules. However, other routes of administration may be possible provided suitable precautions are taken to make the compositions suitable for administration in the contemplated way. For example, the compositions of the invention may be administered parenterally, whether subcutaneously, intravenously, intramuscularly, intrasternally, transdermally or by infusion techniques, or as a suppository.

The following Examples illustrate selected preferred embodiments of the invention.

### Example 1

Copper (II) orotate (35 mg) and manganese (II) orotate (35 mg), in finely divided form were mixed dry. Sodium salicylate solution (3.5 ml of a 10% aqueous solution) was then added followed by vitamin C (400 mg). The resulting suspension is suitable for immediate oral administration.

### Example 2

Copper (II) orotate (35 mg), manganese (II) orotate (35 mg) and zinc orotate (35 mg) in finely divided form were mixed dry. 3.5 ml of a 10% aqueous solution of sodium salicylate (i.e. 3.5 ml of an aqueous solution containing 350 mg sodium salicylate) was then added followed by vitamin C (400 mg). The resulting suspension is suitable for immediate oral administration.

### Example 3

Copper (II) orotate (35 mg), manganese (II) orotate (35 mg), iron (II) orotate (35 mg) and sublimed sulfur (35 mg), in finely divided form were mixed dry. Sodium salicylate solution (3.5 ml of a 10% aqueous solution) was then added followed by vitamin C (400 mg). The resulting suspension is suitable for immediate oral administration.

### Example 4

Copper (II) orotate (35 mg), manganese (II) orotate (35 mg), iron (II) orotate (35 mg), sublimed sulfur (35 mg) and zinc orotate (35 mg) in finely divided form were mixed dry. 3.5 ml of a 10% aqueous solution of sodium salicylate (i.e. 3.5 ml of an aqueous solution containing 350 mg sodium salicylate) was then added followed by vitamin C (400 mg). The resulting suspension is suitable for immediate oral administration.

### Example 5

Copper (II) gluconate (35 mg), vitamin C (400 mg) and manganese (II) gluconate (35 mg), in finely divided form were mixed dry. Sodium salicylate solution (3.5 ml of a 10% aqueous solution) was then added. The resulting solution is suitable for immediate oral administration.

### Example 6

Copper (II) gluconate (35 mg), vitamin C (400 mg), manganese (II) gluconate (35 mg) and zinc gluconate (35 mg) in finely divided form were mixed dry. 3.5 ml of a 10% aqueous solution of sodium salicylate (i.e. 3.5 ml of an aqueous solution containing 350 mg sodium salicylate) was then added. The resulting solution is suitable for immediate oral administration.

### Example 7

Copper (II) gluconate (35 mg), vitamin C (400 mg), manganese (II) gluconate (35 mg), iron (II) gluconate (35 mg) and sublimed sulfur (35 mg), in finely divided form were mixed dry. Sodium salicylate solution (3.5 ml of a 10% aqueous solution) was then added. The resulting suspension is suitable for immediate oral administration.

### Example 8

Copper (II) gluconate (35 mg), vitamin C (400 mg), manganese (II) gluconate (35 mg), iron (II) gluconate (35 mg), sublimed sulfur (35 mg) and zinc gluconate (35 mg) in finely divided form were mixed dry. 3.5 ml of a 10% aqueous solution of sodium salicylate (i.e. 3.5 ml of an aqueous solution containing 350 mg sodium salicylate) was then added. The resulting suspension is suitable for immediate oral administration.

### Example 9

A study was carried out to see if CV 247 (a composition, prepared in accordance with Example 5 above, comprising 2 mg/ml manganese gluconate, 2 mg/ml copper gluconate, 40 mg/ml vitamin C, and 35 mg/ml sodium salicylate) would protect against free radical damage induced by cisplatin during a fourteen-days long oral treatment in rats.

Free radical damage was induced by a single iv dose of 6.5 mg/kg of cisplatin. CV247 was administered orally at dose of 3 ml/kg bd.

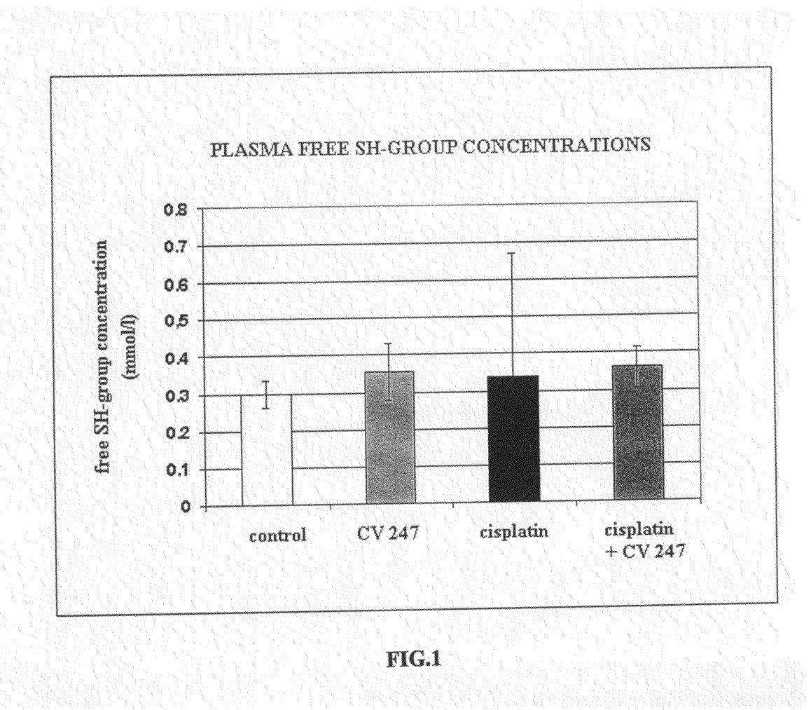

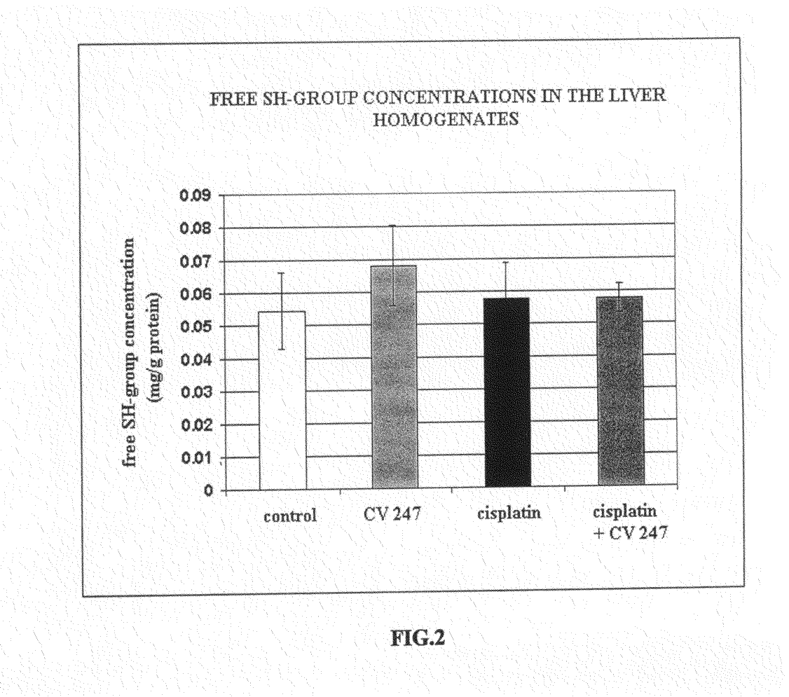

The results found that CV 247 elevated the free SH-group concentration in both in the plasma and liver homogenate of the group of rats not treated with cisplatin, and marginally reduced the harmful effect of cisplatin in the plasma of rats treated with cisplatin (see FIGS. 1 and 2).

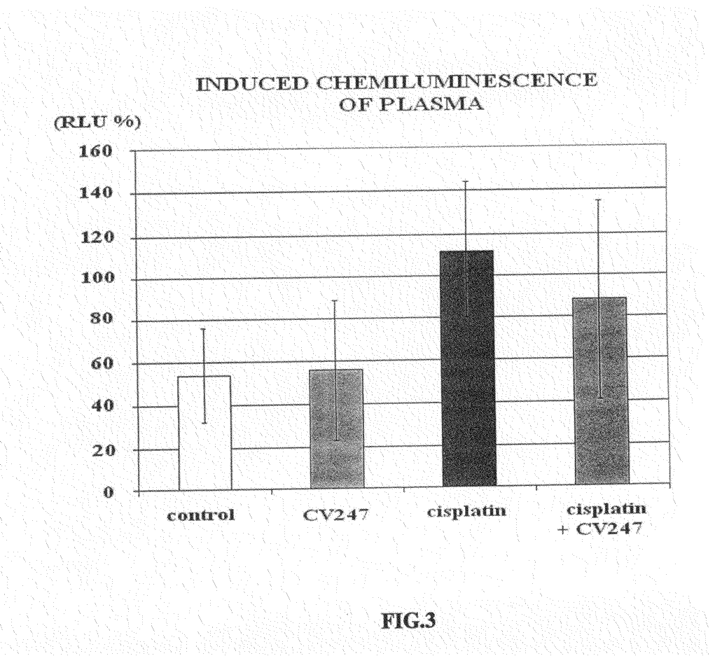

Cisplatin induced free radicals both in the plasma and in the liver (see FIGS. 3 and 4).

CV 247 was able to scavenge the induced free radicals in the liver tissue (FIG. 4). The positive effect of CV 247 can be seen in the plasma as well (FIG. 3).

There was a moderate decrease in diene conjugate concentration during cisplatin treatment, which signals that cisplatin produced lipid peroxidation, which means that diene conjugates were being converted to form malondialdehyde and other lipid peroxidation products. CV 247 inhibited this process (see FIG. 5).

Liver and kidney samples were subsequently examined to see if CV 247 protected the rats from the accumulation of platinum and platinum free radicals, and what kind of metal ion concentration changes occur after treatment.

The cisplatin-only treatment decreased the Cu, Mg, Mn, Mo, P and Zn concentrations in the kidney significantly, and increased the Pb concentration (P<0.05) and the concentration of Pt and Sb become measurable.

The CV247-only treatment decreased Ba, Fe, Mg, P, Se and Zn concentrations in kidney and the concentration of Sb became measurable. The treatment did not change the Cu and Mn concentrations in the kidney, which means that there was no overdose given.

The combined cisplatin and CV247 treatments caused significant changes in Cu, Mn, Mo, Pb, Pt, Se, Si, Sn and Zn concentrations of the kidney (P<0.05) compared to the group treated with cisplatin. The concentration of Cu, Mn, Mo, Se, Si and Zn increased, but of Pb, Pt, Sn decreased. These are positive changes and since the copper transporter Ctrl is highly expressed in basolateral cells of renal tubulus, where it is supposed that the absorption of cisplatin occurs, the effect of Cu treatment in CV247 may result in the down regulation of Ctrl and the consequent inhibition of Pt uptake.

TABLE 1

| | | | | Rat No.s 401-410 |
|---|---|---|---|---|
| | Rat No.s 1-10 | Rat No.s 21-30 | Rat No.s 301-310 | Cisplatin + |
| | Control | Cisplatin | CV247 | CV247 |
| Al | 5.66 ± 1.32 | 6.58 ± 2.60 | 5.71 ± 1.30 | 7.75 ± 2.06 |
| B | <dl | <dl | <dl | 5.85 ± 2.71 |
| Ba | 0.092 ± 0.042 | 0.083 ± 0.058 | 0.057 ± 0.027 | 0.090 ± 0.063 |
| Ca | 67.42 ± 31.12 | 52.82 ± 5.35 | 53.24 ± 16.96 | 60.56 ± 14.38 |
| Co | 0.251 ± 0.036 | 0.210 ± 0.053 | 0.225 ± 0.036 | 0.211 ± 0.054 |
| Cr | 0.135 ± 0.027 | 0.149 ± 0.021 | 0.147 ± 0.059 | 0.138 ± 0.031 |
| Cu | 5.10 ± 0.53 | 3.03 ± 0.81 | 5.07 ± 0.79 | 3.86 ± 0.54 |
| Fe | 42.55 ± 4.01 | 36.20 ± 9.29 | 38.24 ± 2.45 | 38.67 ± 4.29 |
| K | 3948 ± 357 | 3955 ± 281 | 3390 ± 963 | 3794 ± 481 |
| Mg | 152.3 ± 12.1 | 139.0 ± 14.7 | 136.6 ± 15.7 | 151.1 ± 12.6 |
| Mn | 0.789 ± 0.117 | 0.510 ± 0.101 | 0.789 ± 0.083 | 0.598 ± 0.070 |
| Mo | 0.232 ± 0.021 | 0.192 ± 0.026 | 0.253 ± 0.036 | 0.231 ± 0.037 |
| Na | 642.7 ± 82.5 | 657.2 ± 92.2 | 574.3 ± 75.0 | 678.9 ± 120.7 |
| Ni | 0.154 ± 0.061 | 0.135 ± 0.050 | 0.149 ± 0.041 | 0.214 ± 0.108 |
| P | 2502 ± 260 | 2085 ± 265 | 2207 ± 283 | 2283 ± 172 |
| Pb | 1.29 ± 0.73 | 4.36 ± 3.50 | 3.03 ± 1.59 | 1.58 ± 0.72 |
| Pt | <dl | 3.048 ± 0.583 | <dl | 2.074 ± 0.246 |
| S | 1450 ± 124 | 1165 ± 159 | 1364 ± 95 | 1324 ± 106 |
| Sb | <dl | 0.739 ± 0.137 | 0.985 ± 0.318 | 1.000 ± 0.246 |
| Se | 0.191 ± 0.157 | 0.131 ± 0.063 | 0.597 ± 0.470 | 0.294 ± 0.151 |
| Si | 34.78 ± 8.05 | 38.94 ± 5.91 | 38.05 ± 11.62 | 47.79 ± 11.23 |
| Sn | 0.570 ± 0.290 | 1.303 ± 1.076 | 0.860 ± 0.711 | 0.491 ± 0.308 |
| Sr | 0.115 ± 0.116 | 0.064 ± 0.017 | 0.096 ± 0.047 | 0.075 ± 0.045 |
| Zn | 16.10 ± 1.36 | 13.23 ± 1.48 | 14.82 ± 1.20 | 14.95 ± 0.68 |

<dl under detection limit

Cisplatin-only treatment decreased K concentration in plasma significantly and increased B, Co, Cr, Fe, Mg, Mn, Ni and Sb concentrations (P<0.05). The presence of Pb in plasma could also be seen.

The plasma element content did not change in general by the effect of CV247-only treatment. Nevertheless, the concentration of Co and Mg did increase significantly, and the concentration of Pb become measurable in some cases.

The combined cisplatin and CV247 treatment caused a significant decrease of B and Co concentrations compared to cisplatin group (P<0.05). The changes were favorable for Al, Ca, Cr, Fe, K, Mo, Ni, Si and Sr.

The cisplatin-only treatment increased the Fe concentration in liver significantly and Pt appeared in the organ, which may cause an increase in free radical reactions.

The Ca, Mg, Mo and Sr concentrations decreased significantly in the groups treated with CV247-only but the treatment did not change the Cu and Mn concentration in liver.

The combined treatment with cisplatin and CV247 caused significant decreases in Al, B Ca, Co, Cr, Cu, Fe, Mg, Mn, Na, P, Si and Sr concentration compared to cisplatin group (P<0.05). The significant Fe concentration decrease by the CV247 treatment is very favorable, since iron and Fe(II) induced Fenton reaction is responsible for the major part of elevated free radical levels and free radical reactions.

The above data provides strong support for the conclusion that CV247 protects the liver and kidneys from free-radical and heavy metal induced toxicity caused by cisplatin.

### Example 10

The effect of CV247 and cisplatin, alone and in combination, on the viability and cell toxicity of a range of cancer cell cultures was determined in vitro by the following methods:

Sulforhodamine B assay (SRB) to determine cell death and cell viability before and after treatment of CV247. The assay uses the property of SRB to bind with the cytosolic protein and following the determination of absorbance at 570 nm, it is possible to ascertain which cells are dead which are not. It considered highly sensitive method but with the negative feature of lose of linearity on high cell numbers.

Crystal Violet assay (CVE), which though similar to SRB in that it is a protein staining assay, has the advantage of having a more linear relation between optical density and cell numbers.

Tetrazolium dye assay (MTT) is the most widely used determinant of cell number/cell growth. It lacks sensitivity and there are differences in sensitivity between different cell lines, but is useful when considered alongside the other methods used.

The clonogenic assays used in this study are based only on the quantification analysis of living cells using indirect parameters. Individually, these assays have disadvantages. For example The MTT assay is time dependant and is affected by the intracellular concentration of glucose and by pH. The SRB assay is not time dependent, but is based on the fact that sulforhodamine B binds with the intracellular proteins at the amino-end which is an indirect indicator of the cells viability.

All these chemosensitivity assays do not give information quickly about the development of resistant mechanisms or about the metastatic ability of the tumor through neo-angiogenesis. In addition all these assays give only limited information about the malignant phenotype of the cancer and they cannot give solutions about avoiding or reversing the resistance mechanisms.

However, the combination of the above technique provided solid scientific evidence of the effect of the test compounds on a variety of cancer cells, because the advantage of the one technique overlapped the disadvantage of the others, and hence the results were reliable. Hence, collectively, the assays do give very useful information about the potential effectiveness and possible mode of action of new anti-cancer drugs.

The in vitro studies were carried out in a variety of colon (HT55, HCT8 and LoVo) and breast (MFM 223, MDA MB231 and T47D) cancer cell lines. In these studies cisplatin was tested at doses of 1, 5, 10 and 50 mg, the upper dose of which is in excess of that which could ever be used clinically and therefore presented the worst case with regard to its cancer cell toxicity. CV247 was used at a dilution of 1:100. The number of cells per well (18000) represented a medium cancer cell burden.

Taken collectively, the results demonstrated that at a period of up to 48 hr there was a clear enhancement of the anti-neoplastic effect of cisplatin even up to the highly toxic 50 mg dose when combined with CV247, and that this level of effect on cell kill was often replicated when a lower dose of cisplatin was used in combination with CV247. In other words, the extent of cancer cell kill usually only achieved by a very toxic dose of cisplatin could be achieved with a lower dose when combined with CV247.

### Example 11

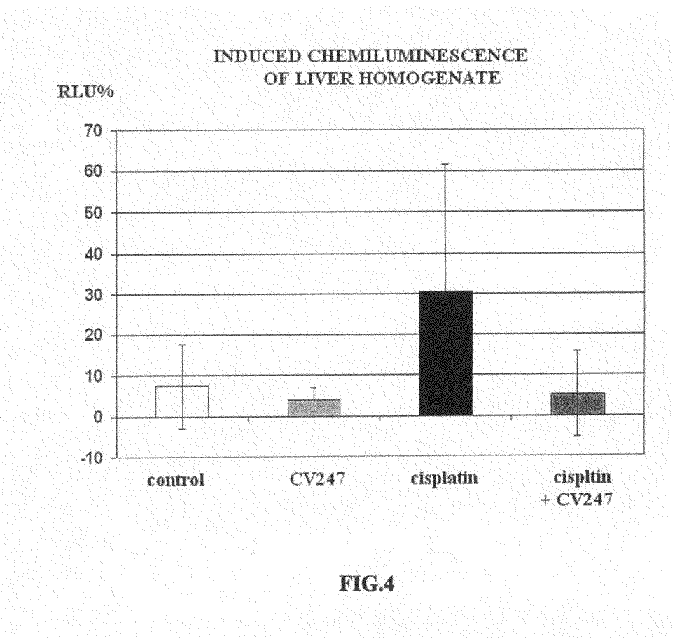

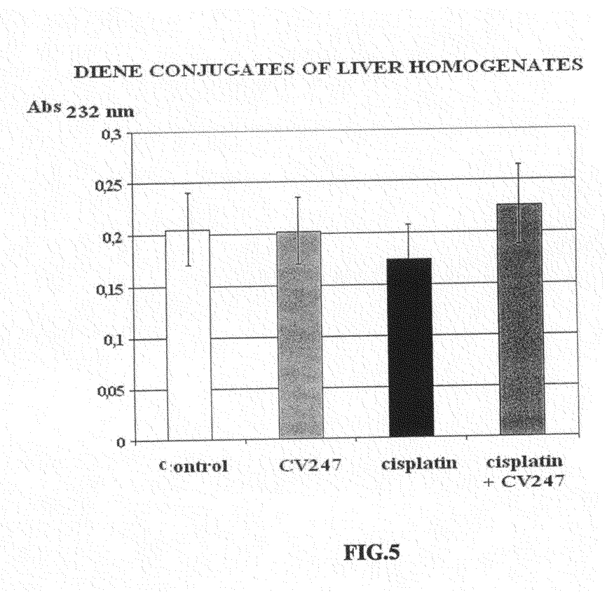

The period during which cisplatin retains its cell kill properties is restricted by the number of doses that can be given clinically, and the fact that after a period of time its effect seems to wane. Thus the positive benefits of combining CV247 with cisplatin observed in Example 10 were further investigated with a longer period of exposure of up to 10 days using both medium (18000) and high (26000) cancer cell burden wells. Graphs depicting the results obtained are shown in FIGS. 6 to 11 (it should be noted that some graphs have the x axis displaying cancer cell kill, whilst others display cancer cell viability).

Though the results varied depending upon the test, there were some consistent observations regardless of the cell line and the test utilized. It is evident that high doses of cisplatin are cytotoxic and that the response to CV247 alone varied regardless of the cancer cell burden. Notably cisplatin at a dose of 1 mg usually had only a marginal long term benefit and that its effect after 4 days was often reduced. But with CV247 that effect was significantly enhanced for 10 days to a level that was only seen with doses of cisplatin alone at least 5 times greater. This data suggests that when combined with compositions such as CV247, cisplatin (and/or other antineoplastic platinum based drugs) could be clinically administered at a dose considerably lower than that which it would be effective if used alone, and that any dose related adverse events could therefore be further reduced (such reduction being additional to the reduced nephrotoxicity provided by the combination of CV247 with cisplatin observed in the rat study described above in Example 9).

It is not clear why CV247 has this effect of enhancing the properties of cisplatin, but one possibility is that it may act as a bridge, enhancing and prolonging the electrostatic effect of cisplatin which helps prevent the strands of DNA in cancer cells opening in the S phase thus preventing the entry of DNA polymerase which is required for DNA duplication in the GII phase (sister chromatid exchange). Alternatively CV247 might act via retro-endoduplication preventing GII entering into the mitotic phase by inhibiting the enzyme involved and resulting in recycling back into GI with consequential hyperdiploidy, a feature associated with increased survival rates in cancer patients.

While aspects of the present invention have been described above, it is to be understood that other similar embodiments may be used or modifications and additions may be made to the described embodiments without departing from the invention. Therefore, the claimed invention should not be limited to any single embodiment, but rather should be construed in breadth and scope in accordance with the appended claims.

1. A method for treating neoplastic disease in a patient, comprising administering to the patient:
(a) an antineoplastic platinum (II) complex;
(b) a physiologically acceptable source of assimilable copper;
(c) a physiologically acceptable source of assimilable manganese;
(d) a source of salicylic acid or a physiologically acceptable derivative thereof; and
(e) vitamin C.

2. The method of claim 1, wherein compound (a) is selected from cisplatin, oxaliplatin and carboplatin.

3. The method of claim 1, wherein compound (a) is cisplatin.

4. The method of claim 1, wherein the method further comprises administering one or more of: (f) a physiologically acceptable source of assimilable iron; (g) a physiologically acceptable source of assimilable zinc; and (h) a physiologically acceptable source of assimilable sulfur.

5. The method of claim 1, wherein compounds (b) and (c) are present in the form of salts of the metal with an organic or inorganic acid.

6. The method of claim 4, wherein compounds (f) and (g), if present, are present in the form of salts of the metal with an organic or inorganic acid.

7. The method of claim 5, wherein the salts are the same or different and are selected from orotates, aspartates, gluconates, tartrates, citrates, lactates and acetates.

8. The method of claim 6, wherein the salts are the same or different and are selected from orotates, aspartates, gluconates, tartrates, citrates, lactates and acetates.

9. The method of claim 4, wherein compound (h), if present, is selected from elemental sulfur and any allotropic form of sulfur.

10. The method of claim 4, wherein compounds (h), if present, is sublimed sulfur.

11. The method of claim 1, wherein compound (d) is selected from salicylic acid or a salicylic acid metal salt, ester or amide.

12. The method of claim 1, wherein compound (d) is sodium salicylate.

13. The method of claim 1, wherein the method comprises administering compound (a) separately from compounds (b)-(e).

14. The method of claim 13, wherein compounds (b)-(e) are administered in a composition in which compounds (b)-(e) are the sole pharmaceutically active components.

15. The method of claim 13, wherein compounds (b)-(e) are administered in a composition further comprising one or more of: (f) a physiologically acceptable source of assimilable iron; (g) a physiologically acceptable source of assimilable zinc; and (h) a physiologically acceptable source of assimilable sulfur.

16. The method of claim 15, wherein the compounds (b)-(e) and the one or more compounds selected form (f)-(g) are the sole pharmaceutically active components in the composition.

17. The method of claim 1, wherein compounds (b)-(e) are administered as a composition comprising:
(b) 15 to 60 parts by weight copper gluconate, or equivalent amount of active ingredient when a physiologically acceptable source of assimilable copper other than copper gluconate is used;
(c) 15 to 60 parts by weight manganese gluconate, or equivalent amount of active ingredient when a physiologically acceptable source of assimilable manganese other than manganese gluconate is used
(d) 300 to 600 parts by weight sodium salicylate, or equivalent amount of active ingredient when salicylic acid or a physiologically acceptable derivative thereof other than sodium salicylate is used; and
(e) 200 to 1000 parts by weight vitamin C;
the parts by weight referred to being based on the total weight of these ingredients in the composition.

18. The method of claim 17, wherein the composition further comprises one or more of:
(f) 15 to 60 parts by weight of iron gluconate, or equivalent amount of active ingredient when a physiologically acceptable source of assimilable iron other than iron gluconate is used;
(g) 15 to 60 parts by weight zinc gluconate, or equivalent amount of active ingredient when a physiologically acceptable source of assimilable zinc other than zinc gluconate is used; and
(h) 15 to 60 parts by weight of sulfur.

19. The method of claim 1, wherein compound (a) is cisplatin, and is administered intravenously at a dose of about 10 to 25 mg/m² every 3 to 4 weeks, or at a dose of about 3 to 4 mg/m² given daily for 5 days, every 3 to 4 weeks.

20. The method of claim 1, wherein the neoplastic disease to be treated is selected from solid malignancies, tumours of the brain, endometrium, oesophagus, stomach, anus, head and neck, and thymus, neuroblastomas, sarcoma of the bone and soft tissue, and carcinomas of the breast, rectum, colon, prostate, bladder, liver, peritoneum, stomach and urethra.

21. A method for enhancing the antineoplastic effect of an antineoplastic platinum (II) complex and/or for reducing the toxic side effects of an antineoplastic platinum (II) complex, comprising administering to a patient receiving said antineoplastic platinum (II) complex:
a physiologically acceptable source of assimilable copper;
a physiologically acceptable source of assimilable manganese;
a source of salicylic acid or a physiologically acceptable derivative thereof; and
vitamin C.

22. An antineoplastic therapeutic combination comprising:
(a) an antineoplastic platinum (II) complex;
(b) a physiologically acceptable source of assimilable copper;
(c) a physiologically acceptable source of assimilable manganese;
(d) a source of salicylic acid or a physiologically acceptable derivative thereof; and
(e) vitamin C.
